(19) United States
(12) Patent Application Publication
(10) Pub. No.: US 2008/0227764 A1
(43) Pub. Date: Sep. 18, 2008

(54) USE OF PREGNANE-DIONES OR DIOLS AS NEUROPATHIC ANALGESIC AGENTS

(76) Inventors: Colin Stanley Goodchild, Melbourne (AU); Raymond Nadeson, Melbourne (AU)

Correspondence Address: KNOBBE MARTENS OLSON & BEAR LLP, 2040 MAIN STREET, FOURTEENTH FLOOR, IRVINE, CA, 92614, US

(21) Appl. No.: 12/041,593

(22) Filed: Mar. 3, 2008

Related U.S. Application Data
(63) Continuation of application No. 10/487,922, filed on Sep. 9, 2004, now abandoned.

(30) Foreign Application Priority Data
Aug. 28, 2001 (AU) PR7319

(51) Int. Cl. A61K 31/57; A61P 25/02

(52) U.S. Cl. 514/178; 514/182

(57) ABSTRACT

The present invention relates to the use of pregnanes in inducing analgesia, preferably without overt sedation, in a mammal in response to neuropathic pain, and compositions and kits therefore.

### CROSS-REFERENCE TO RELATED APPLICATIONS

This application claims the benefit and priority of and is a continuation of U.S. application Ser. No. 10/487,922, filed on Sep. 9, 2004, entitled “USE OF PRGENANE-DIONES OR DIOLS AS NEUROPATHIC ANALGESIC AGENTS,” which is hereby incorporated by reference in its entirety. U.S. application Ser. No. 10/487,922 is a U.S. National Phase of PCT International Application Number PCT/AU02/01144, filed on Aug. 28, 2002, designating the United States of America, which claims priority under 35 U.S.C. § 119 to Australian Application Number PR7319 filed on Aug. 28, 2001.

### BACKGROUND OF THE INVENTION

1. Field of the Invention

The present invention relates generally to methods of inducing analgesia in response to neuropathic pain which involve administration of compounds as shown in formula I, in particular pregnane-diones or pregnane-diols, optionally in association with one or more other analgesic compounds such as opioid compounds. The present invention also relates to compositions and kits useful in inducing analgesia in response to neuropathic pain.

2. Description of the Related Art

The present invention relates generally to the induction of analgesia in response to neuropathic pain. In considering the approaches to treatment of pain it is important to understand the distinction between acute and chronic pain. Acute pain occurs as a result of tissue injury or inflammation and is mediated by chemical, mechanical or thermal stimulation of pain receptors. In contrast, chronic pain in itself constitutes a disease which serves no protective biological function. Chronic pain is unrelenting and can persist for years after an initial injury. Chronic, non-malignant pain predominantly constitutes neuropathic pain which can be defined as pain initiated or caused by a primary lesion or dysfunction within the nervous system¹. Neuropathic pain is associated with a variety of disease states and presents in the clinic with a range of symptoms².

Neuropathic pain is often reported as having a lancinating or continuous burning character and is often associated with the appearance of abnormal sensory signs such as allodynia and hyperalgesia Allodynia is defined as pain resulting from a stimulus which does not normally elicit a painful response and hyperalgesia is characterised by an increased pain response to a stimulus which is normally painful. Some disorders characterised by neuropathic pain include monoradiculopathies, trigeminal neuralgia, postherpetic neuralgia, phantom limb pain, complex regional pain syndromes and the various peripheral neuropathies.

Whereas there are numerous effective therapies for acute pains caused by inflammatory processes or acute injury, especially including treatment with opioid and non-steroidal anti-inflammatory drugs (NSAIDs), neuropathic pain is an area of largely unmet therapeutic need. Due to the distinct pathophysiological mechanisms associated with neuropathic pain relative to inflammatory pains, agents useful in treatment of inflammatory and other pains have reduced effectiveness in neuropathic pain treatment. In particular, the effectiveness of opioids in treatment of neuropathic pain is diminished relative to inflammatory pain treatment and the dose response curve of opioids in neuropathic pain is shifted to the right of that for inflammatory pain⁵. The conventional pharmacological mainstays of clinical management of neuropathic pain are the tricyclic anti-depressants and certain anti-convulsants^{3,4}, but even these achieve clinically significant pain relief (that is greater than 50% pain relief) in less than 50% of patients. These agents are also associated with significant side effect profiles.

There is therefore a pressing need for improved treatment regimes for addressing the problem of neuropathic pain and it is in this context that the present invention has been conceived. Other objects of the present invention will become apparent from the following detailed description thereof

### SUMMARY OF THE INVENTION

According to one embodiment of the present invention there is provided a method of inducing analgesia in response to neuropathic pain in a mammal which comprises administering to the mammal an effective amount of a compound of formula I

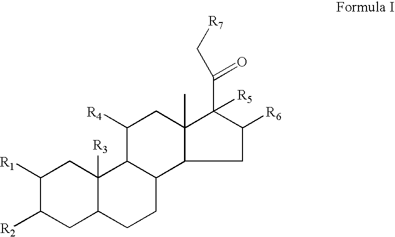

wherein
R₁ is H, OH, C₁-C₄ alkyl, C₂-C₄ alkenyl, C₂-C₄ alkanoyl or —OR;
R₂ is H, OH, OR or ═O;
R₃ is H, OH or C₁-C₄ alkyl;
   R₄ is H, OH, ═O, C₁-C₄ alkyl, C₂-C₄ alkenyl, C₂-C₄ alkanoyl or —OR;
R₅ is H, OH, C₁-C₄ alkyl, C₂-C₄ alkenyl, C₂-C₄ alkanoyl or —OR;
R₆ is H, OH, ═CH₂ or C₁-C₄ alkyl;
   R₇ is H, OH, halogen, C₁-C₄ alkyl, C₂-C₄ alkenyl, C₂-C₄ alkanoyl, SH, SR or —OR;
   and R is C₁-C₄ alkyl, C₂-C₂ alkenyl or C₂-C₄ alkanoyl;

or a pharmaceutically acceptable derivative thereof.

According to another embodiment of the present invention there is provided a method of inducing analgesia, without overt sedation, in response to neuropathic pain in a mammal which comprises administering to the mammal an effective amount of a compound of formula I or a pharmaceutically acceptable derivative thereof.

Another embodiment of the invention provides a composition for inducing analgesia, without overt sedation, in response to neuropathic pain in a mammal comprising a compound of formula I, or a pharmaceutically acceptable derivative thereof, together with at least one pharmaceutically acceptable additive.

In a still further embodiment of the present invention there is provided a method of inducing analgesia in response to neuropathic pain in a mammal which comprises concurrently or sequentially administering to the mammal effective amounts of an analgesic compound, such as an opioid, and a compound of formula I or a pharmaceutically acceptable derivative thereof. Preferably the analgesic compound and compound of formula I, or pharmaceutically acceptable derivative thereof are administered in synergistically effective amounts. Preferably the method does not result in overt sedation.

The invention also relates to the use of a compound of formula I, or a pharmaceutically acceptable derivative thereof, in the manufacture of a medicament for inducing analgesia, preferably without overt sedation, in response to neuropathic pain.

In a still further embodiment of the present invention there is provided a kit for inducing analgesia in response to neuropathic pain in a mammal which comprises an analgesic compound, such as an opioid, and a compound of formula I or a pharmaceutically acceptable derivative thereof.

For example, the analgesic compound may be an opioid selected from one or more of fentanyl, oxycodone, codeine, dihydrocodeine, dihydrocodeinone enol acetate, morphine, desomorphine, apomorphine, pethidine, methadone, dextropropoxyphene, pentazocine, dextromoramide, oxymorphone, hydromorphone, dihydromorphine, noscapine, papaverine, papavereturn, alfentanil, buprenorphine and tramadol and pharmaceutically acceptable derivatives thereof.

In a preferred embodiment, R₇ is OH, OR, SH, SR or halogen, more preferably OH, OR, SH or SR. In another preferred embodiment R₂ is OH or OR, more preferably in the α-conformation.

Preferred compounds of formula I are those wherein

R₁ is H, OH or methyl;

R₂ is OH;

R₃ is H or methyl;

R₄ is H, OH or ═O;

R₅ is H, OH or methyl;

R₆ is H or methyl;

R₇ is OH, OC₂₋₄ alkanoyl (such as OCOCH₃), SH, SCOCH₃, Cl, Br or F.

In another preferred embodiment R₁ is H, R₂ is OH in alpha conformation, R₃ is methyl (in alpha or beta conformation) and R₇ is OH or OR.

In another preferred embodiment, the compound of formula I is a pregnane-dione, i.e. R₂ or R₄ is ═O.

In a particularly preferred embodiment of the invention, the compound according to formula I is alphadolone acetate.

The compounds according to the invention may be administered, inter alia, orally, intravenously, intramuscularly, intraperitoneally, intragastrically, intrathecally. transdermally or intestinally. In a particularly preferred form, the compounds are administered orally.

Preferably the compound according to formula I is administered up to a maximum dose of about 2 grams/70 kg every 6 hours.

In a particularly preferred embodiment of the invention the mammal is a human.

### BRIEF DESCRIPTION OF THE DRAWINGS

The present invention will be further described with reference to the following figures, wherein:

FIG. 1 shows a plot of alphadolone acetate dose (mg/kg) against rest time (seconds) for male Wistar rats intraperitoneally administered 60-200 mg/kg of alphadolone acetate. Results for saline contreated control rats are represented by the broken lines.

FIG. 2 shows a plot of oxycodone dose (mg/kg) against rest time (seconds) for male Wistar rats intraperitoneally administered 0.125-1.0 mg/kg of oxycodone. Results for saline contreated control rates are shown by the broken lines.

FIG. 3 shows a bar graph representation of rest counts for oxycodone (0.5 mg/kg) administered intraperitoneally and oxycodone (0.5 mg/kg) combined with alphadolone acetate (10 mg/kg) also administered intraperitoneally.

FIG. 4 shows a plot of time from drug administration (hours) against withdrawal threshold (log grams) for male Wistar rats subjected to the Chung neuropathy model of L5 and L6 Tight ligation. Paw withdrawal threshold was measured before and after intraperitoneal injection of alphadolone acetate at 20 and 40 mg/kg.

FIG. 5 shows plots of the anti-nociceptive effects in diabetic neuropathic male Wistar rats compared to normal weight matched male Wistar rats following administration of oxycodone (250 μg/kg), alphadolone acetate (a.acetate) (6 mg/kg) or combined oxycodone and alphadolone acetate (a.acetate) at the same doses. Assessment of anti-nociceptive effects was conducted by monitoring noxious electrical current (ACT) (A), tail flick latency (B) and paw pressure (C).

FIG. 6 shows dose response curves for the anti-nociceptive effect of intraperitoneally administered fentanyl in control male Wistar rats (FIG. 6A) and intraperitoneally administered fentanyl given alone and also alphadolone acetate together with fentanyl in diabetic neuropathic male Wistar rats (FIG. 6B).

FIG. 7 shows dose response curves for the anti-nociceptive effect of intraperitoneally administered morphine in control male Wistar rats (FIG. 7A) and intraperitoneally administered morphine given alone and also alphadolone acetate together with morphine in diabetic neuropathic male Wistar rats (FIG. 7B).

FIG. 8 shows dose response curves for the anti-nociceptive effect of intraperitoneally administered oxycodone in control male Wistar rats (FIG. 8A) and of intraperitoneally administered oxycodone given alone and also alphadolone acetate together with morphine in diabetic neuropathic male Wistar rats (FIG. 8B).

FIG. 9 shows dose response curves for the anti-nociceptive effect of intraperitoneally administered alphadolone acetate in control (FIG. 9A) and diabetic neuropathic (FIG. 9B) male Wistar rats.

### DETAILED DESCRIPTION OF THE PREFERRED EMBODIMENT

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As conveyed above, the present invention relates to methods of inducing analgesia in response to neuropathic pain in a mammal. In this context the term “mammal” is intended to encompass both humans and other mammals such as laboratory animals including rats, mice, simians and guinea pigs, domestic animals including cats, dogs, rabbits, agricultural animals including cattle, sheep, goats, horses and pigs and captive wild animals such as lions, tigers, elephants and the like.

Throughout this specification, the term “neuropathic pain” is to be understood to mean pain initiated or caused by a primary lesion or dysfunction within the nervous system. It is the intention of the methods according to the present invention to induce analgesia in response to neuropathic pain being suffered by a mammalian, preferably human, patient. In this context the term “analgesia” is intended to describe a state of reduced sensibility to pain, which preferably occurs without overt sedation and preferably without an effect upon the sense of touch. Preferably, the sensibility to pain is reduced by at least 30%, preferably at least 50%, more preferably at least 70% and particularly preferably at least 85%. In the most preferred aspect of the invention the sensibility to the neuropathic pain is completely, or substantially completely, removed. To assess the level of reduction of sensibility to pain associated with the analgesia induced by the methods according to the present invention it is possible to conduct tests such as the short form McGill pain questionnaire and/or visual analogue scale for pain intensity and/or verbal rating scale for pain intensity and/or measurement of tactile allodynia using von Frey hairs or similar device. These tests are standard tests within the art and would be well known to the skilled person.

By the term “overt sedation” it is intended to convey that the methods (and compositions) of the invention do not result in practically meaningful sedation of the patent, i.e. significant, visible or apparent drowsiness or unconsciousness of the patient being treated. Thus, administration of preferred compounds described herein do not result in sleepiness or drowsiness in the patient to the extent that it interferes with or inhibits the activities associated with day to day living, e.g. driving a motor vehicle or operating machinery for human subjects or feeding and grooming for an animal subject. Where a compound of formula I, or a pharmaceutically acceptable derivative thereof is administered concurrently or sequentially with another analgesic compound, it is to be understood that “overt sedation” refers to sedation over and above any sedation which may be caused by the analgesic compound.

In one embodiment, preferred compounds of Formula I according to the present invention are pregnane-dione compounds. As an example, the chemical structure of 3,20-pregane-dione, together with conventional numbering of the steroidal ring system is shown in Formula II below. Other pregnane-dione compounds contemplated are 11,20-pregnane-diones.

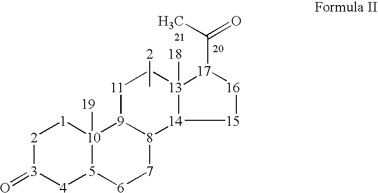

Compounds encompassed by Formula I and related compounds, such as C20 amino pregnanes which may also be useful in the methods described herein, are for example disclosed in The Merck Index (13th Edition) and Australian Patent No. 698,746, U.S. Pat. No. 3,558,608, GB Patent No. 1,317,184, GB Patent No. 1,317,185 as well as German Patent Nos. 2,162,593 and 2,162,554.

The whole of the subject matter of the above documents together with items 105627c and 9285v of Chemical Abstracts, Vol. 77, 1972; 64113v, 64114w, 20793n of Chemical Abstracts 5, Vol. 75, 1971; 115783f and 66672h of Chemical Abstracts Vol. 79, 1973; and 1020345 of Chemical Abstracts Vol. 78, 1973 is to be considered included and imported hereinto.

The documents referred to above provide details of synthetic approaches which may be adopted in production of compounds according to Formula I. Other compounds may be available commercially from sources such as Steraloids (Newport, R.I.) or be naturally occurring. Still other compounds may be obtained by chemical manipulation, using standard methods known in the art, of compounds such as those described in the references above.

A particularly preferred compound according to Formula I is 21-acetoxy-3α-hydroxy-5α-pregnane-11,20-dione which is commonly referred to as alphadolone acetate and is depicted in Formula III.

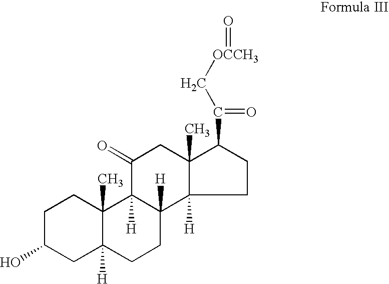

Another compound of formula I contemplated by the invention is the deacylated form of alphadolone acetate:

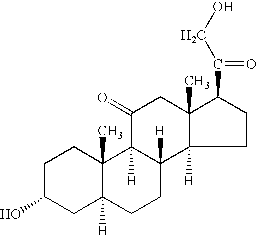

Yet another compound of formula I contemplated by the invention is:

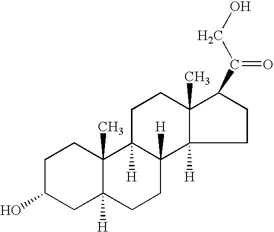

Other preferred compounds according to the invention may include 3α-hydroxy-5α-pregane-11,20-dione; 3α-hydroxy-21-propionyloxy-5α-pregane-11,20-dione; 21-iso-butyryloxy-3α-hydroxy-5α-pregnane-11,20-dione; 21-hemisuccinyloxy-3α-hydroxy-5α-pregane-11,20-dione; 3α-hydroxy-5β-pregnan-20-one; and 3α-hydroxy-5β-pregnan-20-one; (3α-5α)-3,17,21-trihydroxy-pregnan-1,20-dione; (3α-5β)-3,17,21-trihydroxy-0pregnan-11,20-dione; 3β,17βα,21-trihydroxy-5α-pregnan-11,20-dione; 3β,11β,21-trihydroxy-5α-pregnan-11,20-dione; 3β,17βα,21-trihydroxy-5α-pregnan-20-one; 5α-pregnan-3,20-dione and 5β-pregnan-3,20-dione.

Particularly preferred compounds of the invention have an oxy or thio group at the 21-position, e.g. where R₇ is OH, SH, OR (e.g. OC₂₋₄ alkanoyl) or SR, more preferably OH or OR. Other preferred embodiments of the invention are compounds of formula I, or pharmaceutically acceptable derivatives thereof, which are capable of forming a glucuronide metabolite once administered to the patient. Still other preferred compounds are pregnane-dione compounds, e.g. where R₂ or R₄ is ═O. In another embodiment, R₅ and R₆ are hydrogen. In another embodiment, R₇ is OC₂-C₄ alkanoyl. In still another preferred embodiment of the invention, R₇ and R₂ are both independently selected from the group of OH and OR, e.g. R₂ is OH and R₇ is OH or OR. Other preferred compounds are those where R₃ is β-methyl. Particularly preferred compounds of formula I may possess, where appropriate, two or more of the above preferred features.

With reference to Formula I, it will of course be well understood by a person skilled in the art that substitutents not specifically defined, but the presence of which is necessary due to extra valency, will be taken up by hydrogen atoms. It will also be understood in the situation where for example, R₂ represents an oxo radical at the 3 carbon position, there will be no hydrogen bound at this position. This also applies when R₄ is oxo and R₆ is methylene.

The term “alkyl” used herein denotes straight chain, branched or monocyclic alkyl, preferably including from 1-4 carbon atoms. Examples of straight chain, branched and cyclic alkyls include methyl, ethyl, propyl, isopropyl, butyl, cyclopropyl and cyclobutyl.

The term “alkenyl” used herein denotes groups formed from straight chain, branched or cyclic alkenes, including mono- or poly-unsaturated alkyl or cycloalkyl groups. Specific examples include vinyl, allyl, 1-methylvinyl, butenyl and isobutenyl.

The term “alkanoyl” is intended to denote straight or branched chain alkanoyl (COalkyl) groups such as acetyl (COCH₃), propionyl (COCH₂CH₃), butyryl (COCH₂CH₂CH₃) and isobutyryl (COCH(CH₃)₂).

In another aspect of the present invention the method of inducing analgesia in response to neuropathic pain may involve concurrent or sequential administration to the mammal in need of such treatment of additively, or more preferably, synergistically effective amounts of a compound of formula I, or a pharmaceutically acceptable derivative thereof, and another analgesic compound such as an opioid. Thus, a synergistically effective amount of a compound of formula (I), or pharmaceutically acceptable derivative thereof, when administered concurrently or sequentially with an opioid may restore opioid responsiveness to neuropathic pain. The compound of formula I, or pharmaceutically acceptable derivative thereof, and the opioid may be administered either as a combined form, i.e. a single composition containing the active agents, or as discrete dosages. The active agents are temporally administered such that the desired additive or synergistic analgesic effect is achieved.

As used herein, opioid compounds (opioids) include any compound which is a partial or full agonist of an opioid receptor.

Opioid compounds are well known and include naturally occurring compounds derived from opium such as codeine, morphine and papaverine as well as derivatives of such compounds which generally have structural similarity and other compounds which are active as analgesic agents. Specific examples of opioid compounds contemplated by the present invention may include: fentanyl, oxycodone, codeine, dihydrocodeine, dihydrocodeinone enol acetate, morphine, desomorphine, apomorphine, pethidine, methadone, dextropropoxyphene, pentazocine, dextromoramide, oxymorphone, hydromorphone, dihydromorphine, noscapine, papaverine, papavereturn, alfentanil, buprenorphine and tramadol and pharmaceutically acceptable derivatives and/or tautomers thereof.

The phrase “pharmaceutically acceptable derivative” is intended to convey any pharmaceutically acceptable salt, pro-drug, hydrate, solvate, metabolite or any other compound which, upon administration to the subject, is capable of providing (directly or indirectly) the compound concerned or a physiologically (e.g. analgesically) equivalent active compound, or an active metabolite or residue thereof. An example of a suitable derivative is an ester formed from reaction of an OH or SH (e.g. C21 OH or SH) group with a suitable carboxylic acid, for example C₁₋₃alkyl-CO₂H, and HO₂C—(CH₂)ₙ—CO₂H (where n is 1-10, preferably 14), and CO₂H—CH₂ phenyl.

Thus, the compounds of formula I may be in crystalline form, either as the free compounds or as solvates (e.g. hydrates). Methods of solvation are generally known within the art.

The salts of the active compounds of the invention are preferably pharmaceutically acceptable, but it will be appreciated that non-pharmaceutically acceptable salts also fall within the scope of the present invention, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulfphonic, toluenesulphonic, benzenesulphonic, salicyclic, sulphanilic, aspartic, glutamic, edetic, stearic, palmitic, oleic, lauric, pantothenic, tannic, ascorbic and valeric acids.

The term “pro-drug” is used herein in its broadest sense to include those compounds which can be converted in vivo to the compound of interest (e.g. by enzymatic or hydrolytic cleavage). Examples thereof include esters, such as acetates of hydroxy or thio groups, as well as phosphates and sulphonates. Processes for acylating hydroxy or thio groups are known in the art, e.g. by reacting an alcohol (hydroxy group), or thio group, with a carboxylic acid. Other examples of suitable pro-drugs are described in Design of Prodrugs, H. Bundgaard, Elsevier, 1985.

The term “metabolite” includes any compound into which a compound of formula I can be converted in vivo once administered to the subject. Examples of such a metabolite are a glucuronide, a sulphate and hydroxylates.

It will be understood that the compounds as described herein may exist in a tautomeric form to that depicted, i.e. as a tautomer thereof. The term “tautomer” is used herein in its broadest sense to include compounds which are capable of existing in a state of equilibrium between two isomeric forms. Such compounds may differ in the bond connecting two atoms or groups and the position of these atoms or groups in the compound. A specific example is keto-enol tautomerism.

The compounds of the invention may be electrically neutral or be polycations with associated anions for electrical neutrality. Suitable associated anions include sulfate, tartrate, citrate, chloride, nitrate, nitrite, phosphate, perchlorate, halosulfonate or trihalomethylsulfonate.

Neuropathic pain which may be treated by the methods of the invention include monoradiculopathies, trigeminal neuralgia, postherpetic neuralgia, phantom limb pain, complex regional pain syndromes, neuropathic pain associated with AIDS and infection with the human immunodeficiency virus and the various peripheral neuropathies, including, but not limited to drug-induced and diabetic neuropathies.

The compounds of Formula I, and pharmaceutically acceptable thereof, and the analgesic compounds (e.g. opiates) which may be optionally administered in conjunction with them (referred to as the “active ingredients, agents or compounds”) may be administered for therapy by any suitable route. It will be understood that compounds of formula I or their derivatives are preferably administered via a route which does not result in overt sedation of the subject. Suitable routes of administration may include oral, rectal, nasal, inhalation of aerosols or particulates, topical (including buccal and sublingual), transdermal, vaginal, intravesical and parenteral (including subcutaneous, intramuscular, intravenous, intrasternal, intrathecal, epidural and intradermal). Preferably, administration of a compound of formula I or a pharmaceutically acceptable derivative thereof will be by a route which when administered first presents the compound to the stomach of the subject. In a particularly preferred embodiment of the invention, the compound of formula I is administered via an oral route, however it will be appreciated that the preferred route will vary with the condition and age of the subject, the nature of the neuropathic pain being treated, its location within the subject and the judgement of the physician or veterinarian.

As used herein, an “effective amount” refers to an amount of active compound which provides the desired analgesic activity when administered according to a suitable dosing regime. Preferably the amount of a compound of formula I, or pharmaceutically acceptable derivative thereof is an amount which provides the desired analgesic activity without causing overt sedation. Dosing may occur at intervals of minutes, hours, days, weeks or months. Suitable dosage amounts and regimes can be determined by the attending physician or veterinarian. For example, compounds of formula I, or pharmaceutically acceptable derivatives thereof, may be administered to a subject at a rate of 50 to 2000 mg every six hours, such as 50-500 mg. Dosing of the analgesic agent, such as an opioid, can be determined by the attending physician in accordance with dosing rates in practice. For example, fentanyl can be administered in an amount of about 100 μg whereas morphine may be administered in an amount of 1-5 grams.

The compositions of the present invention comprise at least one compound of Formula I or pharmaceutically acceptable derivative thereof, optionally with an analgesic compound such as an opioid, together with one or more pharmaceutically acceptable additives such as carriers, diluents adjuvants and/or excipients and optionally other medicaments. These include all conventional solvents, dispersion agents, fillers, solid carriers, coating agents, antifungal or antibacterial agents, dermal penetration agents, surfactants, isotonic and absorption agents and slow or controlled release matrices. Compositions for use in the present invention may also include other supplementary physiologically active agents, e.g. other analgesic agents. The compounds may be presented in the form of a kit of components which is adapted for allowing concurrent or sequential administration of the active components. Each carrier, diluent, adjuvant and/or excipient must be pharmaceutically “acceptable” in the sense of being compatible with the other ingredients of the composition and not injurious to the subject. The compositions may conveniently be presented in unit dosage form and may be prepared by methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

Compositions of the present invention suitable for oral administration may be presented as discrete units such as capsules, sachets or tablets each containing a predetermined amount of the active ingredient; as a powder or granules; as a solution or a suspension in an aqueous phase or non-aqueous liquid; or as an oil-in-water liquid emulsion or a water-in-oil emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent, preservative disintegrant (e.g. sodium starch glycollate, cross-linked povidone, cross-linked sodium carboxymethyl cellulose) surface-active or dispersing agent. Moulded tablets may be made my moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspended agents and thickening agents. The compositions may be presented in a unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Compositions suitable for topical administration to the skin, i.e. transdermal administration, may comprise the active compounds dissolved or suspended in any suitable carrier or base and may be in the form of lotions, gels, creams, pastes, ointments and the like. Suitable carriers may include mineral oil, propylene glycol, waxes polyoxyethylene, and long chain alcohols. Transdermal devices, such as patches may also be used and may comprise a microporous membrane made from suitable material such as cellulose nitrate/acetate, propylene and polycarbonates. The patches may also contain suitable skin adhesive and backing materials.

The compounds of formula I may also be presented as implants which may comprise a drug bearing polymeric device wherein the polymer is biocompatible and non-toxic. Suitable polymers may include hydrogels, silicones, polyethylenes and biodegradable polymers.

The compounds of the invention may be administered in a sustained (i.e. controlled) or slow release form. A sustained release preparation is one in which the active ingredient is slowly released within the body of the subject once administered and maintains the desired drug concentration over a minimum period of time. The preparation of sustained release formulations is known to the skilled person. Dosage forms may include oral forms, implants and transdermal forms. For slow release administration, the active ingredients may be suspended as slow release particles or within liposomes

It should be understood that in addition to the ingredients particularly mentioned above, the composition of this invention may include other agents conventional in the art having regard to the type of composition in question, for example, those suitable for oral administration may include such further agents as binders, sweetners, thickeners, flavouring agents, disintegrating agents, coating agents, preservatives, lubricants and/or time delay agents.

Other details of pharmaceutically acceptable carriers, diluents and excipients and methods of preparing pharmaceutical compositions and formulations are provided in Remmingtons Pharmaceutical Sciences 18^{th} Edition, 1990, Mack Publishing Co., Easton, Pa., USA, the disclosure of which is included herein in its entirety by way of reference.

The compounds for use in the invention may also be presented for use in veterinary compositions. These may be prepared by any suitable means known in the art. Examples of such compositions include those adapted for
(a) oral administration, e.g. drenches including aqueous and non-aqueous solutions or suspensions, tablets, boluses, powders, granules, pellets for admixture with feedstuffs, pastes for application to the tongue;
(b) parenteral administration, e.g. subcutaneous, intramuscular or intravenous injection as a sterile solution or suspension;
(c) topical application, e.g. creams, ointments, gels, lotions, etc.

Particularly preferred compounds according to Formula I include alphadolone acetate and alphadolone glucuronide or other pro-drug which will provide a 21-OH group in vivo. If other salt forms of active agents according to Formula I are adopted it is preferred to adopt either sulfate or methane sulfonate salts, more preferably at the C21 position.

In a particularly preferred embodiment of the invention the compound according to Formula I is administered orally, preferably in the form of a tablet, capsule, lozenge or liquid. The administered composition will preferably include a surfactant and/or solubility improver. A suitable solubility improver is water-soluble polyethoxylated caster oil and an example of a suitable surfactant is Cremophor EL. Dose ranges suitable for alphadolone or the pregnane-diones are for example 50 to 500 mg orally, every six hours. Suitable dose ranges for morphine are 2.5 to 20 mg every 3 to 6 hours and for oxycodone and other opioids 2 to 50 mg every 3 to 12 hours.

### EXAMPLES

The present invention will now be further described with reference to the following examples which are intended for the purpose of illustration only and are not intended to limit the generality hereinbefore described.

### Example 1

### Animal Models of Neuropathic Pain

### Experimental Approach

There are no human experimental neuropathic pain models. There are several animal models that differ in the method of induction of pain and in the relative balance of signs and symptoms. Thus researchers, in attempting to identify a useful pharmacotherapy, will use a battery of these models.

The majority of neuropathic pain models in current use share as a common feature alterations in hind-limb cutaneous sensory thresholds following partial injury of a peripheral (usually sciatic) nerve. In particular, demonstration of hyperalgesia to noxious thermal stimuli and allodynia to cold and mechanical stimuli are used as outcome measures. Two of the most commonly used models are the chronic constriction injury (CCI) of sciatic nerve⁷ and the spinal nerve ligation model (SNL)⁶. The CCI model consists of the loose ligation of the sciatic nerve at mid-thigh level with chromic gut sutures⁷. An inflammatory reaction develops in response to the catgut and consequentially a loss of most A-fibres and some C-fibres, but few cell bodies⁸. This is associated with spontaneous pain-related behaviour, allodynia and hyperalgesia. The SNL model (Chung model) consists of injury to the L5 and L6 spinal nerves, which contribute to the sciatic nerve⁶. Once again, this is associated with the development of spontaneous pain-like behaviour as well as long lasting allodynia and hyperalgesia.

The majority of animal models of neuropathy have been based on a discrete peripheral nerve injury. However, some have been developed to more closely mimic individual disease states. An example of this is the streptozotocin model of peripheral diabetic neuropathy⁹. In this model, injections of streptozotocin induce diabetes and then hyperalgesia and allodynia.

In the present investigations the sedative effects of alphadolone acetate and an opioid, oxycodone, were investigated when the drugs were given alone and also together. Non sedative doses so identified were tested for analgesic efficacy in two rat neuropathic pain models, the Chung model of tight L5/6 nerve root ligation, and streptozotocin-induced diabetic neuropathy. Alphadolone acetate was used as an example of analgesic neurosteroids that have the unique property of analgesic properties by an action on spinal cord GABA_{A} receptors and oxycodone was chosen to typify the behaviour and results expected of a range of opioid drugs used clinically.

### (i) Examination of Sedation Effects

Male Wistar rats (n=10 per dosage group) were given a range of doses of alphadolone acetate (60 to 200 mg/kg intraperitoneally in 10% Cremophor EL) or oxycodone (0.125 to 1.0 mg/kg intraperitoneally in saline) alone and also oxycodone (0.5 mg/kg) at the same time as a dose of alphadolone acetate (10 mg/kg, both intraperitoneally). These rats were naive to the drugs and also to the open field activity monitor. After the injection of drug or drug combination the activity of each rat was monitored by the breaking of a grid of intersecting infrared beams in a dark box. The resting time calculated from the number of beam breaks were counted for 30 minutes. The ten replicate observations for each drug dose were combined and means +/−SEM plotted on dose response curves alongside saline controls.

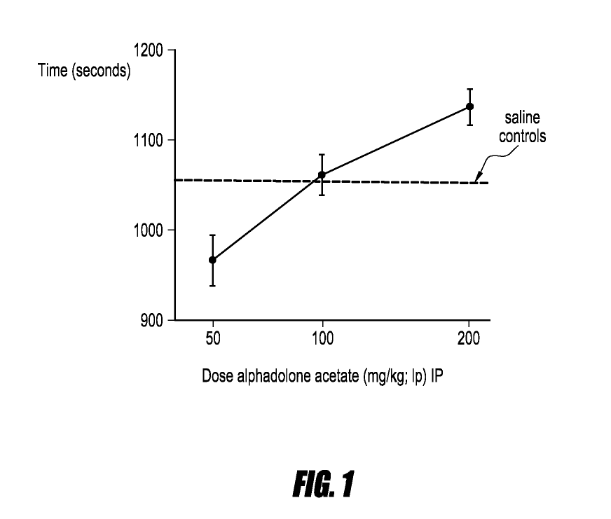

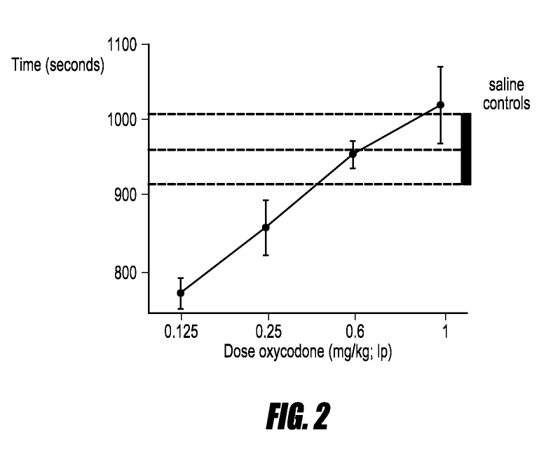

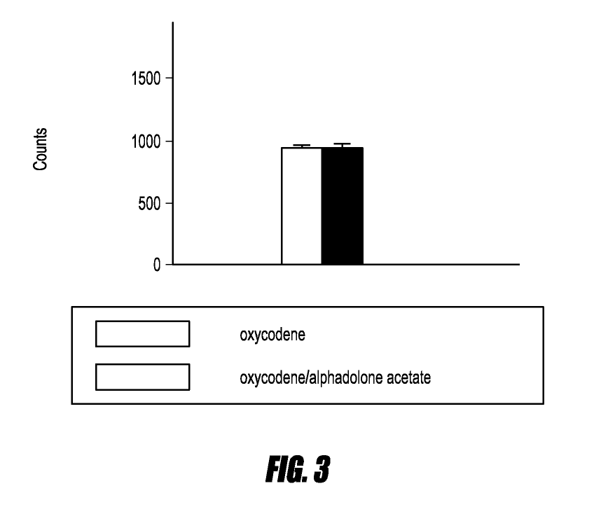

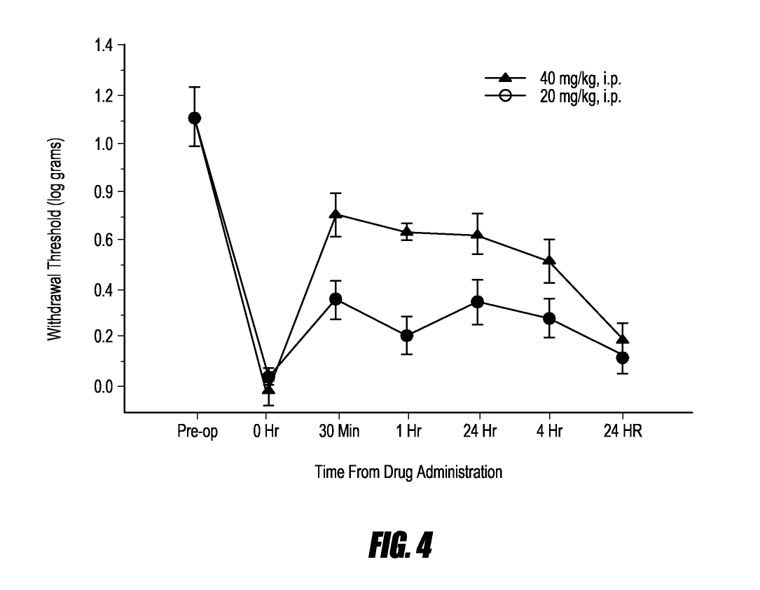

The results for resting times in the experiments using the open field activity monitor are shown below for alphadolone acetate and oxycodone given alone compared with saline controls (FIGS. 1 and 2) and for oxycodone 0.5 mg/kg given alone and with alphadolone acetate 10 mg/kg (FIG. 3).

Doses of alphadolone acetate less than 100 mg/kg and oxycodone less than 1.0 mg/kg did not increase resting times compared with saline controls. Lower doses of each drug (alphadolone acetate 60 mg/kg; oxycodone 0.25 and 0.125 mg/kg) actually decreased resting times compared with saline controls. Furthermore the addition of 10 mg/kg alphadolone acetate to 0.5 mg/kg oxycodone did not cause any increase in sedation assessed by resting time in the open field activity monitor. Thus any anti-nociceptive activity observed in neuropathic pain models that these doses or lower of each drug given alone or in combination might exhibit could not be explained by the occurrence of sedation.

### (ii) Chung Neuropathic Pain Model

Rats were prepared with tight nerve root ligations according to the method described by Kim and Chung⁶. The paw withdrawal thresholds were measured before and after intraperitoneal injections of alphadolone (20 and 40 mg/kg; n=10 per group). This was achieved using a Randall Sellito apparatus in which an increasing weight was applied to the neuropathic paw. The paw withdrawal threshold was the weight at which the rat withdrew its paw from the apparatus.

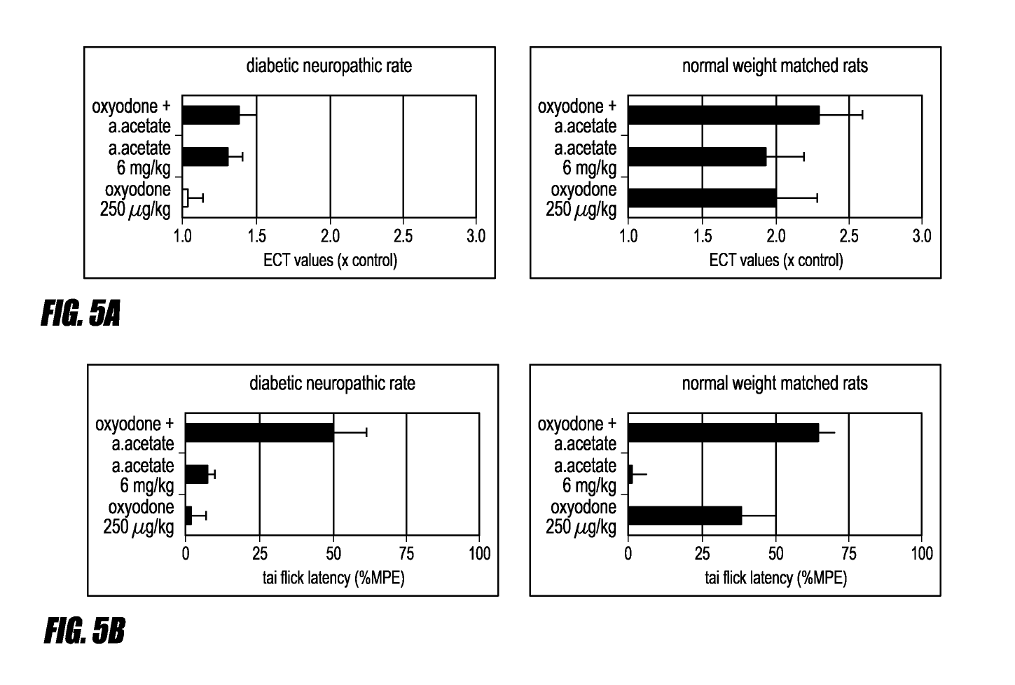

This is a model of neuropathic pain in which allodynia is induced in one hind paw of the rat by tight ligatures placed on the ipsilateral lumbar dorsal rootlets. The results in this model for groups of 10 rats that received alphadolone acetate intraperitoneally are shown in FIG. 4. It can be seen that the nerve ligation reduces the threshold of withdrawal from a mechanical stimulus from normal nociceptive stimulus levels pre-op to levels that are normally non-nociceptive at time 0 when alphadolone was administered.

(iii) Diabetic Neuropathic Pain Model

Seventy rats were injected intraperitoneally with streptozotocin (STZ) (150 mg/kg total dose) (Sapphire Bioscience) dissolved in sodium chloride (0.9%). The 150 mg dose was given in two 75 mg/kg injections on consecutive days. An additional group of animals was injected with saline to act as a vehicle control (n=10). Diabetes was confirmed one week after injection of STZ by measurement of tail vein blood glucose levels with Ames Glucofilm test strips and a reflectance colorimeter (Ames Glucometer 3, Bayer Diagnostics). Only animals with final blood glucose levels L 15 mM were deemed to be diabetic. The rats were retested for hyperglycaemia immediately after each session of nociceptive testing. Hyperalgesia was assessed using the paw pressure test, previously described by Randall and Selitto (Randall L. O, Selitto, J. J. 1957 A Method for Measurement of Analgesic Activity in Inflamed Tissue Archiv. Int. Pharmacodynamie: 111; 409). Replicate results in each group were combined to calculate means +/−SEM that were plotted as histograms. Tests took place 5 weeks after the first injection of STZ. Animals that had paw pressure nociceptive thresholds below 30 g (60% of the value in normal weight matched rats) were deemed to have developed hyperalgesia/neuropathic pain and thus used in further experiments. This was 91% of all STZ treated rats.

The anti-nociceptive effects of alphadolone acetate (6 mg/kg ip) and oxycodone (250 μg/kg) each given alone and in combination to groups of normal weight matched and diabetic neuropathic rats (n=10 per group) were assessed with noxious electrical current (ECT), tail flick latency (TFL) and paw pressure.

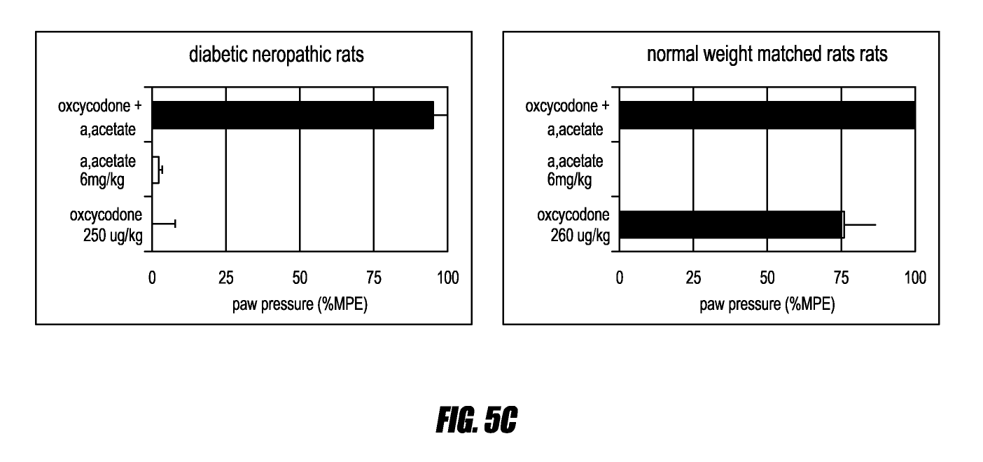

The anti-nociceptive effects assessed with noxious electrical current (ECT), tail flick latency (TFL) and paw pressure of alphadolone acetate (6 mg/kg ip) and oxycodone (250 μg/kg) each given alone and in combination to groups of normal weight matched and diabetic neuropathic rats are shown in FIGS. 5 (A, B, and C respectively).

It can be seen that diabetic neuropathic pain is minimally responsive to the anti-nociceptive action of the opioid or alphadolone acetate when either drug is used alone. However, the combination of both drugs led to anti-nociception equal in magnitude to that obtained in normal rats with the opioid. The doses of the neurosteroid and opioid used alone or the combination were well below those that cause sedation as assessed by the open field activity monitor. Importantly, the anti-nociceptive effect shown when opioid and alphadolone acetate were administered together was greater than that expected from addition of their individual effects.

### Example 2

### Model for Neuropathic Pain

Courteix and co-workers have developed a diabetes-induced model for neuropathic pain.

They found that induction of experimental insulin-dependent diabetes mellitus in rats caused allodynia and hyperalgesia¹⁰. They went on to show that intravenous morphine induced a dose-dependent anti-nociceptive effect at doses twice as high as those in normal rats using the mechanical nociceptive paw pressure test¹⁰. Thus the diabetic model reproduced the experience of diabetic neuropathic pain in humans; it is opioid resistant. The experiments reported here use this model to assess the relative efficacy of alphadolone acetate and three opioids, fentanyl, morphine and oxycodone given alone and in combinations in causing anti-nociception assessed with paw pressure measured using the Randall Sellito method.

### Methods:

Male Wistar rats (wt 65-80 g) were used for these experiments. Animals were housed 5 per cage under standard laboratory conditions. Food and water were provided ad libitum.

### (i) Induction of Diabetes/Hyperalgesia

Rats were injected intraperitoneally (IP) with streptozotocin (STZ) (150 mg/kg total dose) (Sapphire Bioscience) dissolved in sodium chloride (0.9%). The 150 mg dose was given in two 75 mg/kg injections on consecutive days. Diabetes was confirmed one week after injection of STZ by measurement of tail vein blood glucose levels with Ames Glucofilm test strips and a reflectance colorimeter (Ames Glucometer 3, Bayer Diagnostics). Only animals with final blood glucose levels □15 mM were deemed to be diabetic. The rats were retested for hyperglycaemia once per week to confirm continued high blood glucose readings. Hyperalgesia was assessed using the paw pressure test, previously described by Randall and Selitto¹¹.

Tests took place 5 weeks after the first injection of STZ. Animals that had paw pressure nociceptive thresholds below 30 g (60% of the value in normal weight matched rats) were deemed to have developed hyperalgesia/neuropathic pain and thus used in further experiments. This was 25% of all STZ treated rats in this series of experiments.

### (ii) Nociceptive Tests

After the successful documentation of the development of hyperalgesia in diabetic animals by the paw pressure test, more extensive nociceptive testing paradigms were carried out in diabetic neuropathic animals and weight-matched controls; the control rats were 1-2 weeks younger. Paw pressure (PP) was measured by the method described by Randall and Selitto¹¹ using a Ugo-Basile Algesimeter (Apelex; probe 1 mm; weight: log; increasing pressure to the left hind paw was applied until vocalisation was elicited. The following protocol was used in each experiment in groups of rats (n=4-8) with diabetic neuropathy and normal weight matched controls:
paw withdrawal thresholds measured every 5 minutes for 15 minutes to give readings a, b, and c
intraperitoneal injection of drug or drug combination
paw withdrawal thresholds measured every 5 minutes for a further 35 minutes to give readings d, e, f, g, h, i, and j

The starting thresholds a, b and c varied between individual rats. Thus, in order to obtain meaningful results the responses to the intraperitoneal drugs were standardised according to the equation below in neuropathic rats.

$$\% maximum effect = \frac{( mean of h , i , j ) - ( mean of a , b , c )}{( X ) - ( mean of a , b , c )} \times 100$$

and standardised according to the equation below in normal weight matched controls

$$threshold increase as ratio of starting level = 1 + \frac{( mean of h , i , j ) - ( mean of a , b , c )}{( X )}$$

in which X represents the mean of all predrug paw pressure thresholds in weight matched normal rats. In normal weight matched rats, a response so calculated of 2.0 would indicate that the drug treatment doubled the nociceptive threshold for paw pressure. In diabetic neuropathic rats a 100% response so calculated means that the drug or drug combination elevated the paw withdrawal threshold to the threshold found in normal rats; the allodynia and hyperalgesia was reversed totally. Results for replicate experiments with a particular drug or drug combination at each dose were combined and expressed as means and SEM.

The experiments were performed in a blinded fashion i.e. the person performing the measurements of paw pressure thresholds was unaware of the doses of drugs given. Dose response curves were constructed for each of the opioids given alone in normal weight matched control rats and also for each of the opioids given alone and in combination with 1.0 mg/kg intraperitoneal alphadolone acetate in rats with diabetic neuropathy. In addition dose response curves were constructed for alphadolone acetate given alone in normal weight matched control rats as well as rats with diabetic neuropathy.

(iii) Results

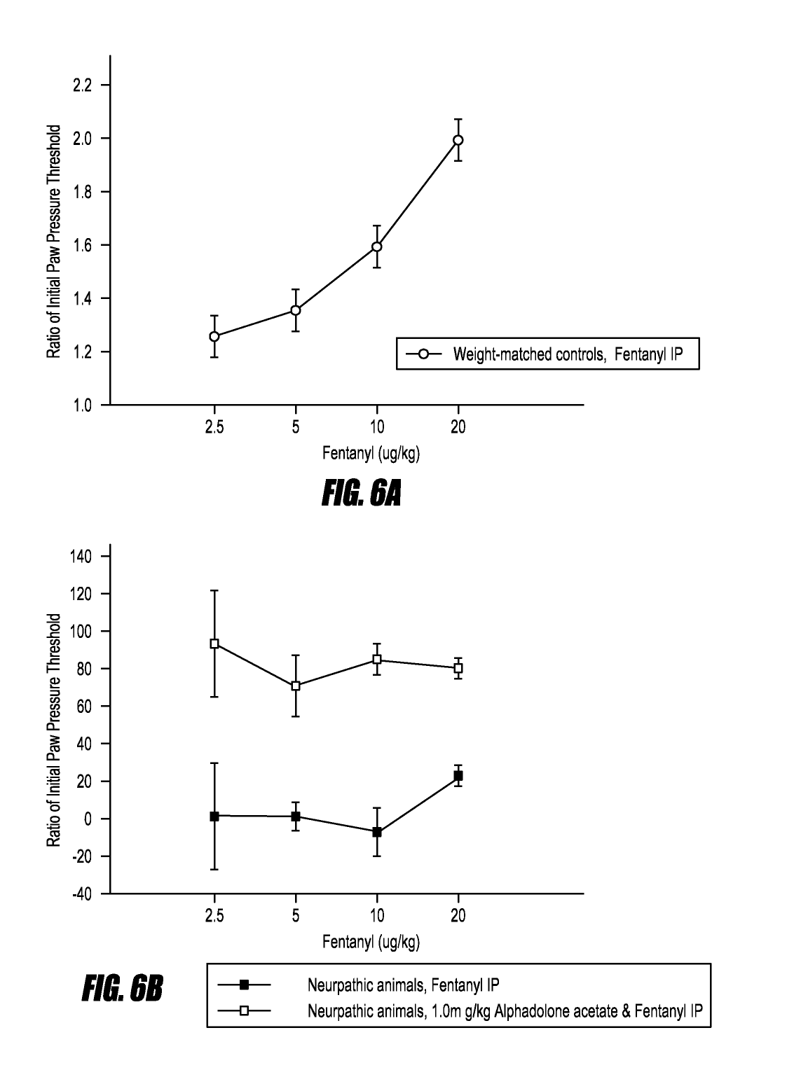

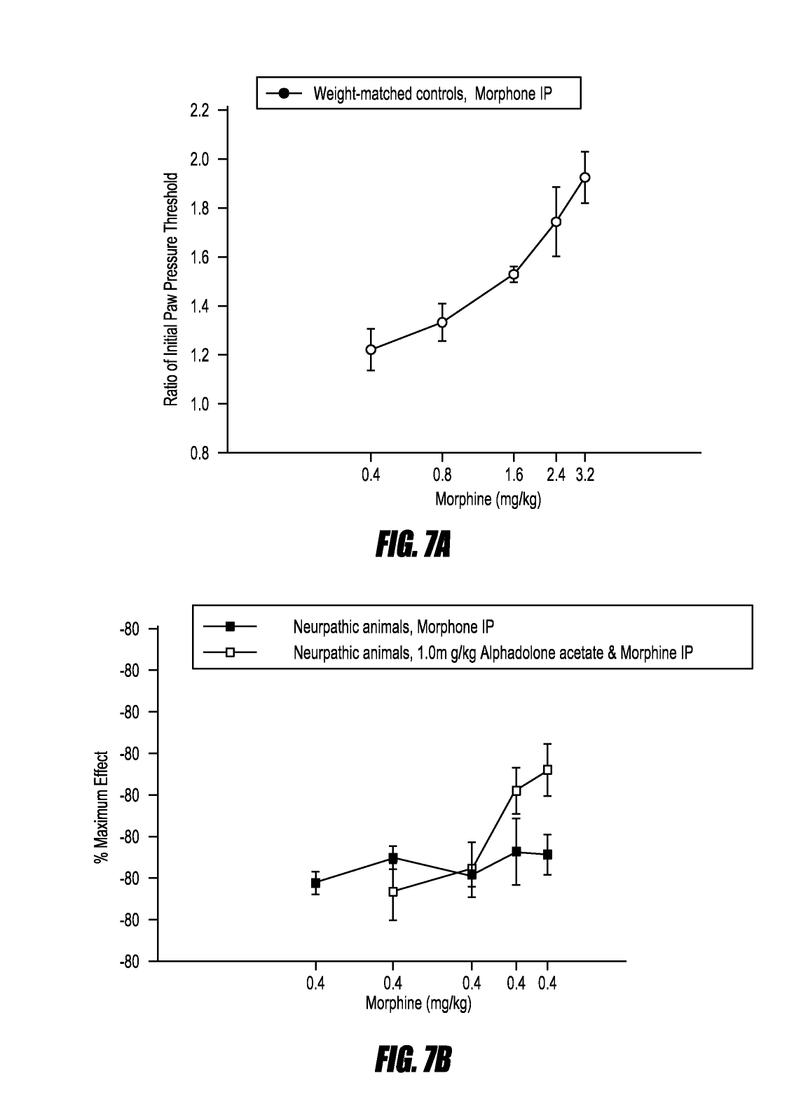

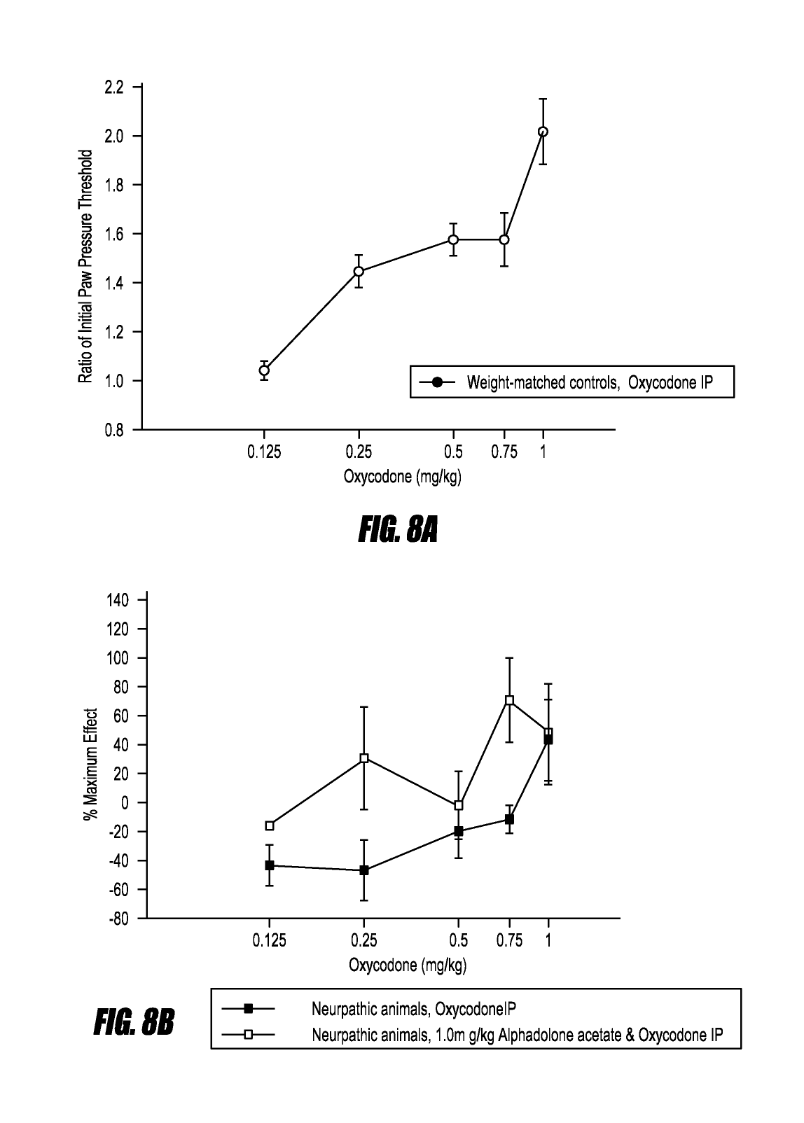

FIGS. 6A, 7A and 8A show dose response curves for each of 3 opioids in normal rats and in diabetic neuropathic rats. In all cases doses of opioids that caused significant antinociception in normal weight matched rats caused little or no reversal of the allodynia and hyperalgesia in rats with diabetic neuropathy.

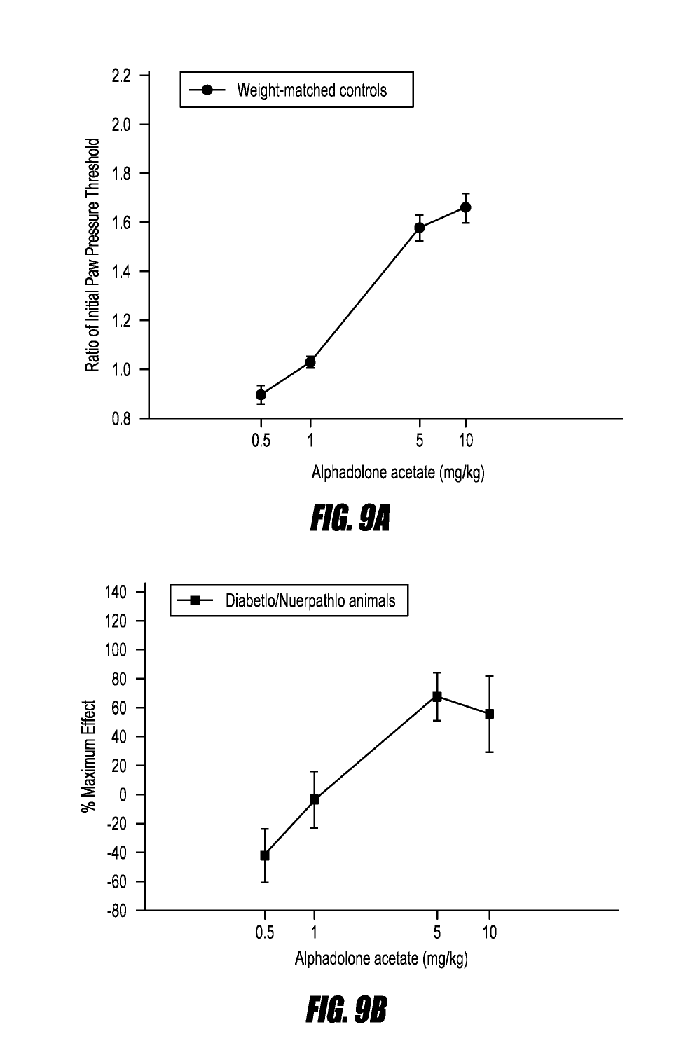

Alphadolone acetate, on the other hand, as can be seen in FIG. 9, caused dose related anti-nociceptive effects in rats with diabetic neuropathy with the same potency as for anti-nociceptive responses in normal weight matched rats. The two dose response curves overlay each other. The maximum dose of alphadolone acetate used in these studies (10 mg/kg) reversed 80% of the allodynia and hyperalgesia to paw pressure in diabetic neuropathic rats.

When 1.0 mg/kg alphadolone acetate, which is ineffective in causing anti-nociception when given alone, was coadministered with each of the opioids in diabetic neuropathic rats, significant anti-nociception occurred (FIGS. 6B, 7B, and 8B) Doses of opioids that were ineffective when given alone completely reversed the allodynia and hyperalgesia to paw pressure in diabetic neuropathic rats.

It should be understood that the present invention has been described by way of example only and that modifications and/or alterations thereto which would be apparent to a skilled person based upon the disclosure herein are also considered to fall within the scope and spirit of the invention.

### REFERENCES

1. Merskey H, Bogduk N. Classification of Chronic Pain, 2nd edn. Seattle: IASP Press, 1994; 394
2. Woolf C J and Mannion R J. Pain: neuropathic pain: aetiology, symptoms, mechanisms and management. Lancet 1999; 353: 1959-64
3. McQuay H J, Tramer M, Nye B A, Carroll D, Wiffen P J, Moore R A. A systematic review of antidepressants in neuropathic pain. Pain 1996; 68: 217-27
4. Sindrup S H, Jensen T S. Efficacy of pharmacological treatments of neuropathic pain: an update and effect related to mechanism of drug action. Pain 1999; 83: 389-400
5. Rowbotham M C. Kalso E, McQuay H J, Wiesenfeld-Hallin Z, eds. The debate over opioids and neuropathic pain. Opioid Sensitivity of Chronic Non-cancer Pain. Seattle: IASP Press, 1999; 18: 307-17
6. Kim S H, Chung J M. An experimental model for peripheral neuropathy produced by segmental spinal nerve ligation in the rat. Pain 1992; 50: 355-63
7. Bennett G J, Xie Y K. A peripheral mononeuropathy in rat that produces disorders of pain sensation like those seen in man. Pain 1988; 33: 87-107
8. Tandrup T, Woolf C J, Coggeshall R E. Delayed loss of small dorsal root ganglion cells after transection of the rat sciatic nerve. J Comp Neurol 2000; 422: 172-80
9. Malcangio M, Tomlinson D R. A pharmacologic analysis of mechanical hyperalgesia in streptozotocin/diabetic rats. Pain 1998; 76: 151-7
10. Courteix, C., Eschalier, A., and Lavarenne, J., Streptozocin-induced diabetic rates: behavioural evidence for a model of chronic pain, Pain, 1993, 63, 81-88.
11. Randell, L. O., and Selitto, J. J., A method for measurement of analgesic activity of inflamed tissue, Archiv. Inst. Pharmacdynamie, 1957, 111, 409.

What is claimed is:

1. A method of inducing analgesia, comprising:
identifying mammal suffering from or at risk of suffering from neuropathic pain; and
administering to said mammal an effective amount of a compound of formula I 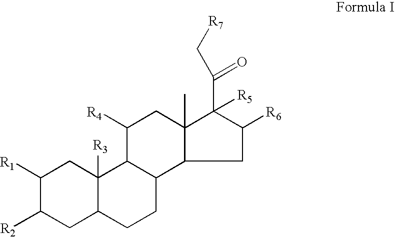
wherein
R₁ is H, methyl, OH, C₁-C₄ alkyl, C₂-C₄ alkenyl, C₂-C₄ alkanoyl or —OR;
R₂ is H, OH, OR or ═O;
R₃ is H, methyl, OH or C₁-C₄ alkyl;
R₄ is H, OH, ═O, C₁-C₄ alkyl, C₂-C₄ alkenyl, C₂-C₄ alkanoyl or —OR;
R₅ is H, methyl, OH, C₁-C₄ alkyl, C₂-C₄ alkenyl, C₂-C₄ alkanoyl or —OR;
R₆ is H, methyl, OH, ═CH₂ or C₁-C₄ alkyl; and
R₇ is H, OH, OCOCH₃, halogen, C₁-C₄ alkyl, C₂-C₄ alkenyl, C₂-C₄ alkanoyl, SH, SCOCH₃, SR or —OR
or a pharmaceutically acceptable derivative thereof.

2. The method of claim 1, wherein
R₁ is H or methyl;
R₂ is OH;
R₃ is H or -methyl;
R₄ is ═O;
R₅ is H or methyl;
R₆ is H or methyl; and
R₇ is OH, OCOCH₃, SH, or SCOCH₃.
